(19) United States
(12) Patent Application Publication
(10) Pub. No.: US 2019/0269309 A1
(43) Pub. Date: Sep. 5, 2019

(54) ELECTRONIC SCOPE AND ELECTRONIC ENDOSCOPE SYSTEM

(71) Applicant: HOYA CORPORATION, Tokyo (JP)

(72) Inventor: Kunihiko ONOBORI, Tokyo (JP)

(73) Assignee: HOYA CORPORATION, Tokyo (JP)

(21) Appl. No.: 16/308,455

(22) PCT Filed: Aug. 24, 2017
(86) PCT No.: PCT/JP2017/030383

§ 371 Date: Dec. 7, 2018

(30) Foreign Application Priority Data
Sep. 1, 2016 (JP) 2016-170601

(51) Int. Cl. A61B 1/07; G02B 23/26; A61B 1/06

(52) U.S. Cl. CPC A61B 1/07; A61B 1/0646; A61B 1/0684; G02B 23/26

(57) ABSTRACT

An electronic scope capable of preventing reduction in the amount of light in a desired wavelength band is configured to be inserted into a body cavity, and includes: an insertion tube having a light emission port in its tip portion; a light guide configured to guide light down to the tip portion of the insertion tube to emit first light from the tip portion; and a light emitting device configured to emit second light from the tip portion, the light transmittance of the light guide for the wavelength band of the second light being less than or equal to the transmittance for the wavelength band of the first light. The light path length of the second light from the light emitting device to an emission port for the second light provided in the tip portion is shorter than the light path length of the first light in the light guide.

### TECHNICAL FIELD

The present invention relates to an electronic scope and an electronic endoscope system.

### BACKGROUND ART

An endoscope system that changes the spectral intensity characteristic of irradiation light to enable shooting of a special image is known. For example, Patent Document 1 describes a concrete configuration of a light source device used in an endoscope system of this type.

The endoscope system described in Patent Document 1 includes a light source device provided with two light emitting diodes (LEDs) and an optical filter. One of the two LEDs is a violet LED that emits light in the violet wavelength band. The other LED is a fluorescent LED that has a blue LED and a yellow fluorescent material. By mixing blue LED light and yellow fluorescence, pseudo-white light is emitted. The optical filter is a wavelength selective filter that transmits only light in a specific wavelength band and is placed insertably/extractably on a light path of irradiation light emitted from the fluorescent LED.

In the light source device described in Patent Document 1, when the optical filter has been extracted from the light path, a subject is irradiated with light emitted from the fluorescent LED as white light without limitation in the wavelength band. When the optical filter has been inserted to the light path, the subject is irradiated with both of light emitted from the fluorescent LED in a limited wavelength band and light emitted from the violet LED. In this way, by changing the spectral intensity characteristic of the irradiation light to irradiate the subject with only light in a specific wavelength band, it is possible to obtain a shot image that enhances a specific tissue of the subject in a living body.

### CITATION LIST

Patent Document
Patent Document 1: WO 2012/108420

### SUMMARY OF INVENTION

### Problems to be Solved by the Invention

In the endoscope system described in Patent Document 1, light emitted from the light source device is input into an optical fiber in an electronic scope. The light guided through the optical fiber is output from the tip portion of the electronic scope. The optical fiber has a characteristic of transmitting light in the visible light band, but has wavelength dependence of the transmittance according to its material. For example, quartz generally used for the optical fiber is lower in transmittance as the wavelength of light is shorter. Therefore, when a subject is observed using violet light comparatively short in wavelength, the amount of the violet light is small causing a problem that the resultant shot image is dark. Also, the optical fiber becomes yellow depending on the material used and due to degradation over time in some cases. Because of this yellowing, there has been a problem that the transmittance of the optical fiber for light in the violet wavelength band degrades, making the shot image further dark.

The above problems arise from the fact that the light transmittance of the optical fiber is different among the wavelength bands.

In view of the above problems, the present invention is directed to provide an electronic scope and an electronic endoscope system capable of preventing reduction in the amount of light in a desired wavelength band even though the optical fiber has a characteristic that its light transmittance is different among the wavelength bands.

### Means for Solving the Problems

In order to solve the above problems, the electronic scope according to an embodiment of the present invention includes:

an insertion tube configured to be inserted into a body cavity and provided with a light emission port in its tip portion;

a light guide configured to guide light down to the tip portion of the insertion tube so as to emit first light from the tip portion; and

a light emitting device configured to emit second light from the tip portion, the light transmittance of the light guide for the wavelength band of the second light being less than or equal to the transmittance for the wavelength band of the first light.

The light path length of the second light from the light emitting device to an emission port for the second light placed in the tip portion is shorter than the light path length of the first light in the light guide.

According to an embodiment, the light emitting device is preferably placed in the tip portion.

According to an embodiment, the second light preferably has a peak wavelength between a wavelength of 405 nm and a wavelength of 425 nm.

According to an embodiment, preferably, a plurality of solid-state light emitting devices are placed in the tip portion, and the light emitting device is one of the plurality of solid-state light emitting devices.

According to an embodiment, the electronic scope preferably includes a light source device configured to emit the first light to the light guide.

The electronic endoscope system according to an embodiment of the present invention includes:

the electronic scope described above; and

an electronic endoscope processor to which the electronic scope is removably connectable.

The electronic endoscope processor includes

the light source device configured to emit the first light, and

a light source drive circuit configured to generate a control signal that controls light emission of the light emitting device and the light source device.

According to an embodiment, the electronic endoscope processor preferably has an optical filter insertable to and extractable from the light path of the first light.

According to an embodiment, the optical filter preferably has a filter characteristic of transmitting only light in the green wavelength band out of the visible light band.

The electronic endoscope system according to another embodiment of the present invention includes:

the electronic scope described above; and

a light source device configured to emit the first light to the light guide.

According to an embodiment, the first light preferably includes light having a wavelength longer than the second light.

According to an embodiment, the light source device preferably has a plurality of light source units configured to emit light different in wavelength band from each other.

According to an embodiment, one of the plurality of light source units is preferably a light source unit configured to emit the second light.

According to an embodiment, preferably, the electronic endoscope system described above includes a light source drive circuit configured to generate a control signal for individually controlling light emission of the light emitting device and the light source device according to each of a plurality of modes, and

the light source drive circuit is configured to generate a first control signal that drives at least the light source device to emit light in a first mode and generate a second control signal that drives at least the light emitting device to emit light in a second mode, thereby controlling the light source device and the light emitting device.

According to an embodiment, preferably, the electronic endoscope system described above includes a light source drive circuit configured to generate a control signal for individually controlling light emission of the solid-state light emitting devices and the light source device,

the electronic scope includes an imaging device configured to image a subject with a predetermined frame period to generate an image signal, and

the light source drive circuit is configured to generate a first control signal that drives at least the light source device to emit light and a second control signal that drives at least the light emitting device to emit light alternately switching between the control signals for each frame of the image signal.

### Advantageous Effects of the Invention

According to the electronic scope and the electronic endoscope system described above, reduction in the amount of light in a desired wavelength band can be prevented even though the optical fiber has a characteristic that its light transmittance is different among the wavelength bands.

### BRIEF DESCRIPTION OF DRAWINGS

FIG. 1 is a block diagram of an electronic endoscope system according to an embodiment of the present invention.

FIG. 2 is a block diagram of a light source device according to the first embodiment of the present invention.

FIGS. 3(a) and 3(b) are views showing spectral intensity distributions of illumination light according to the first embodiment of the present invention.

FIG. 4 is a block diagram of a light source device according to the second embodiment of the present invention.

FIGS. 5(a) and 5(b) are views showing spectral intensity distributions of illumination light according to the second embodiment of the present invention.

FIG. 6 is a block diagram of a light source device according to the third embodiment of the present invention.

FIGS. 7(a) and 7(b) are views showing spectral intensity distributions of illumination light according to the third embodiment of the present invention.

### DESCRIPTION OF EMBODIMENTS

Embodiments of the present invention will be described hereinafter with reference to the accompanying drawings. Note that an electronic endoscope system provided with an endoscope-specific light source device will be described hereinafter as an embodiment of the present invention.

An electronic scope of an embodiment of the present invention includes:

an insertion tube configured to be inserted into a body cavity and provided with a light emission port in its tip portion;

a light guide configured to guide light down to the tip portion of the insertion tube so as to emit first light from the tip portion; and

a light emitting device configured to emit second light from the tip portion, the light transmittance of the light guide for the wavelength band of the second light being less than or equal to the transmittance for the wavelength band of the first light.

In relation to the above, the light path length of the second light from the light emitting device to an emission port for the second light placed in the tip portion is shorter than the light path length of the first light in the light guide that guides the first light.

As described above, since the light path length of the second light, for which the light transmittance of the light guide is less than or equal to the transmittance for the wavelength band of the first light, is shorter than the light path length of the light guide that guides the first light, light loss of the second light due to the light guide is not incurred at all, or reduced, irrespective of whether or not the second light is guided by the light guide. Thus, reduction in the amount of the second light emitted from the tip portion as illumination light can be completely prevented or reduced. According to an electronic scope of an embodiment, the light emitting device is preferably placed in the tip portion of the electronic scope. By doing so, guidance of the second light by the light guide can be made unnecessary, and thus light loss of the second light due to the light guide will not occur at all.

According an electronic scope of an embodiment, the second light may be emitted from the emission port in the tip portion using guidance by a light guide cable. In this case, also, since the length of the light guide cable that guides the second light is short, the light loss of the second light due to the light guide will be reduced compared to the case of being guided by the same light guide cable as the first light.

According an embodiment, the light emitting device is preferably configured to emit the second light for which the light transmittance of the light guide is lower than the transmittance for the wavelength band of the first light.

Embodiments of the present invention will be described hereinafter.

### First Embodiment

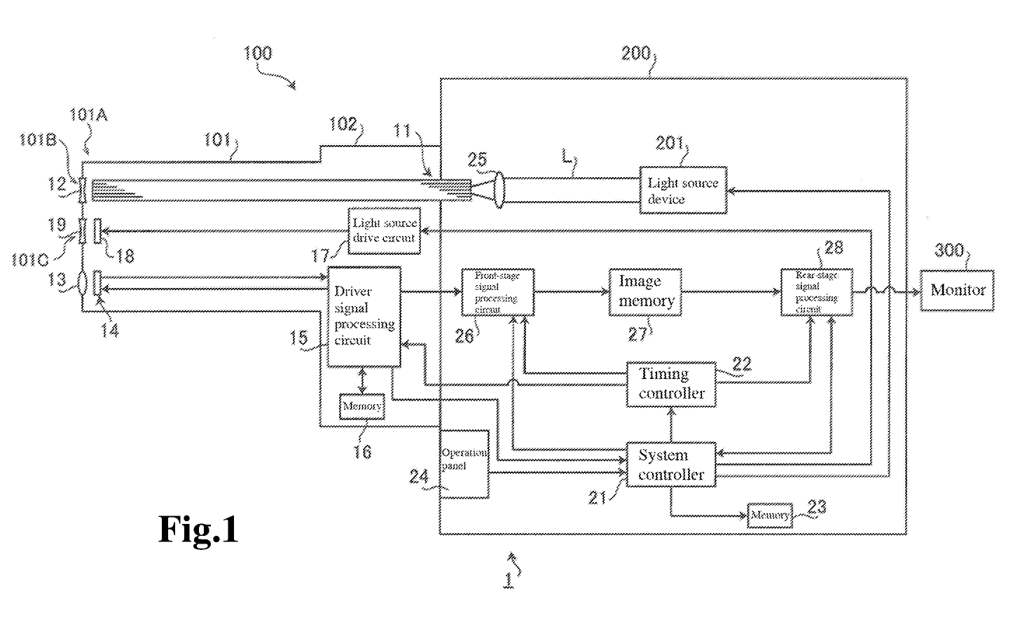

FIG. 1 is a block diagram showing a configuration of an electronic endoscope system 1 provided with an endoscope-specific light source device 201 according to the first embodiment of the present invention. As shown in FIG. 1, the electronic endoscope system 1, a system specialized for medical use, includes an electronic scope 100, a processor 200, and a monitor 300.

The electronic scope 100 has an insertion tube 101 that is inserted into a human body cavity and a connecting portion 102. The electronic scope 100 is removably connected to the processor 200 via the connecting portion 102.

The processor 200 includes a system controller 21 and a timing controller 22. The system controller 21 executes various programs stored in a memory 23 and controls the entire electronic endoscope system 1 in an integrated manner. Also, the system controller 21 is connected to an operation panel 24. In response to instructions from the operator input via the operation panel 24, the system controller 21 changes the operations of the electronic endoscope system 1 and parameters for the operations. The timing controller 22 outputs clock pulses for adjusting the timing of the operations of the components to the circuits in the electronic endoscope system 1.

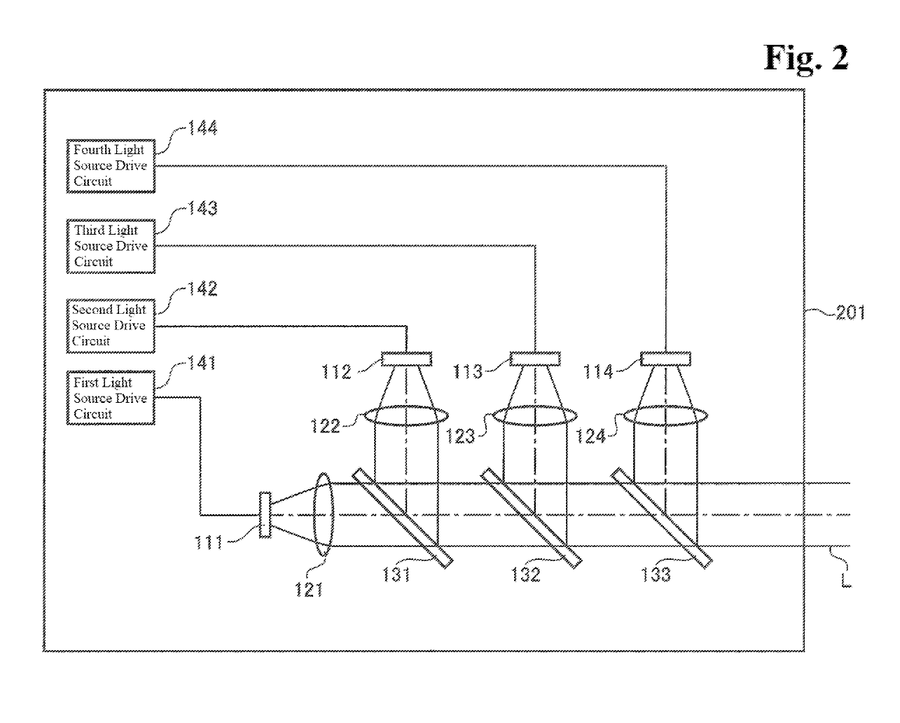

The processor 200 includes a light source device 201. FIG. 2 shows a block diagram of the light source device 201. The light source device 201 includes first to fourth light source units 111 to 114. Light emission of the first to fourth light source units 111 to 114 is individually controlled by control signals generated by first to fourth light source drive circuits 141 to 144, respectively.

The first light source unit 111 is a red light emitting diode (LED) that emits light in the red wavelength band (e.g., wavelengths of 620 to 680 nm). The second light source unit 112 has a blue LED that emits light in the blue wavelength band (e.g., wavelengths of 430 to 470 nm) and a fluorescent material. The fluorescent material is excited by the blue LED light emitted from the blue LED, to emit fluorescence in the green wavelength band (e.g., wavelengths of 460 to 600 nm). The third light source unit 113 is a blue LED that emits light in the blue wavelength band (e.g., wavelengths of 430 to 470 nm). The fourth light source unit 114 is a violet LED that emits light in the violet wavelength band (e.g., wavelengths of 395 to 435 nm).

Collimator lenses 121 to 124 are placed forward of the light source units 111 to 114, respectively, in the light emitting direction. The red LED light emitted from the first light source unit 111 is converted to parallel light by the collimator lens 121 and incident to a dichroic mirror 131. The light emitted from the second light source unit 112, i.e., the blue LED light and the green fluorescence are converted to parallel light by the collimator lens 122 and incident to the dichroic mirror 131. The dichroic mirror 131 combines the light path of the light emitted from the first light source unit 111 and the light path of the light emitted from the second light source unit 112. Specifically, the dichroic mirror 131, having a cut-off wavelength near a wavelength of 600 nm, has a characteristic of transmitting light having a wavelength longer than or equal to the cut-off wavelength and reflecting light having a wavelength shorter than the cut-off wavelength. Therefore, the red LED light emitted from the first light source unit 111 passes through the dichroic mirror 131, and the light emitted from the second light source unit 112 is reflected from the dichroic mirror 131. In this way, the light path of the red LED light and the light path of the blue LED light and the green fluorescence are combined. The light of which the light paths have been combined by the dichroic mirror 131 is incident to a dichroic mirror 132.

The blue LED light emitted from the third light source unit 113 is converted to parallel light by the collimator lens 123 and incident to the dichroic mirror 132. The dichroic mirror 132 combines the light path of the light incident from the dichromic mirror 131 and the light path of the blue LED light emitted from the third light source unit 113. Specifically, the dichroic mirror 132, having a cut-off wavelength near a wavelength of 500 nm, has a characteristic of transmitting light having a wavelength longer than or equal to the cut-off wavelength and reflecting light having a wavelength shorter than the cut-off wavelength. Therefore, the red LED light and the green fluorescence, out of the light incident from the dichroic mirror 131, pass through the dichroic mirror 132, and the blue LED light is reflected from the dichroic mirror 132. Also, the blue LED light emitted from the third light source unit 113 is reflected from the dichroic mirror 132. In this way, the light path of the red LED light and the green fluorescence and the light path of the blue LED light emitted from the third light source unit 113 are combined. The light of which the light paths have been combined by the dichroic mirror 132 is incident to a dichroic mirror 133.

The violet LED light emitted from the fourth light source unit 114 is converted to parallel light by the collimator lens 124 and incident to the dichroic mirror 133. The dichroic mirror 133 combines the light path of the light incident from the dichromic mirror 132 and the light path of the violet LED light emitted from the fourth light source unit 114. Specifically, the dichroic mirror 133, having a cut-off wavelength near a wavelength of 430 nm, has a characteristic of transmitting light having a wavelength longer than or equal to the cut-off wavelength and reflecting light having a wavelength shorter than the cut-off wavelength. Therefore, the light path of the light incident from the dichroic mirror 132 and the light path of the violet LED light emitted from the fourth light source unit 114 are combined by the dichroic mirror 133, and the resultant light is emitted from the light source device 201 as illumination light L.

The illumination light L emitted from the light source device 201 is collected to the incident end face of a light carrying bundle (LCB) 11 by a condenser lens 25 and input into the LCB 11.

The illumination light L input into the LCB 11 propagates through the LCB 11. The illumination light L having propagated through the LCB 11 is output from the outgoing end face of the LCB 11 located in a tip portion 101A of the electronic scope 100 to irradiate a subject via a light distribution lens 12 placed at an emission port 101B. Return light from the subject irradiated with the illumination light L from the light distribution lens 12 forms an optical image on a light receiving surface of a solid-state imaging device 14 via an objective lens 13.

The solid-state imaging device 14 is a single-plate color charge coupled device (CCD) image sensor having a Bayer pixel array. The solid-state imaging device 14 accumulates an optical image formed on each pixel of the light receiving surface as charge corresponding to the light amount, generates red (R), green (G), and blue (B) pixel signals, and outputs the generated signals. Note that the solid-state imaging device 14 is not necessarily a CCD image sensor, but may be replaced with a complementary metal oxide semiconductor (CMOS) image sensor or any other kind of imaging device. The solid-state imaging device 14 may otherwise be one including complementary filters.

A driver signal processing circuit 15 is provided in the connecting portion 102 of the electronic scope 100. Image signals of the subject are input into the driver signal processing circuit 15 from the solid-state imaging device 14 with a predetermined frame period. The frame period is 1/30 seconds, for example. The driver signal processing circuit 15 executes predetermined processing for the image signals received from the solid-state imaging device 14, and outputs the resultant signals to a front-stage signal processing circuit 26 of the processor 200.

The driver signal processing circuit 15 also accesses a memory 16 and reads unique information of the electronic scope 100. Examples of the unique information of the electronic scope 100 recorded in the memory 16 include the number of pixels and sensitivity of the solid-state imaging device 14, the operable frame period, and the model number. The driver signal processing circuit 15 outputs the unique information read from the memory 16 to the system controller 21.

The system controller 21 performs various types of computation based on the unique information of the electronic scope 100, to generate control signals. Using the generated control signals, the system controller 21 controls the operations and timing of various circuits in the processor 200 so that suitable processing be performed for the electronic scope 100 connected to the processor 200.

The timing controller 22 supplies clock pulses to the driver signal processing circuit 15 according to the timing control by the system controller 21. The driver signal processing circuit 15 controls the drive of the solid-state imaging device 14 at the timing synchronizing with the frame period of an image processed on the processor 200 side according to the clock pulses supplied from the timing controller 22.

The front-stage signal processing circuit 26 executes predetermined signal processing, such as de-mosaicing, matrix computation, and Y/C separation, for the image signals received from the driver signal processing circuit 15 with a frame period, and outputs the resultant signals to an image memory 27.

The image memory 27 buffers the image signals received from the front-stage signal processing circuit 26, and outputs the signals to a rear-stage signal processing circuit 28 according to the timing control by the timing controller 22.

The rear-stage signal processing circuit 28 processes the image signals received from the image memory 27, to generate screen data for monitor display, and converts the generated screen data for monitor display to a predetermined video format signal. The converted video format signal is output to the monitor 300. In this way, an image of the subject is displayed on the screen of the monitor 300.

Also, an LED 18 (the light emitting device or the solid-state light emitting device) is placed in the tip portion 101A of the insertion tube 101 of the electronic scope 100. Light emission of the LED 18 is controlled by a control signal generated by a light source drive circuit 17 placed in the connecting portion 102. The LED 18 is a violet LED that emits light in the violet wavelength band (e.g., wavelengths of 395 to 435 nm). A subject is irradiated with the violet LED light emitted from the LED 18 via a light distribution lens 19 placed at an emission port 101C. The LED 18 is placed in the tip portion 101A to prevent light emitted from the LED 108 (the second light) from being guided by the LCB 11 that absorbs part of the light. The LCB 11 has a transmission characteristic that the transmittance is different among the wavelength bands of light. Therefore, the LED 18 emits light in a wavelength band for which the light transmittance of the light guide is less than or equal to the transmittance for the wavelength band of the light emitted from the light source device 201. In this embodiment, the light emitted from the LED 18 is light in the violet wavelength band.

The electronic endoscope system 1 of an embodiment has a plurality of observation modes including a normal observation mode and a special observation mode. These observation modes can be switched to each other depending on the subject to be observed manually or automatically. For example, when it is desired to observe the subject under illumination with normal light, the observation mode is switched to the normal observation mode. The normal light is white light or pseudo-white light, for example. White light has a flat spectral intensity distribution in the visible light band. Pseudo-white light is not flat in spectral intensity distribution, but is a mixture of light components in a plurality of wavelength bands. Also, when it is desired to obtain a shot image where a specific body tissue has been enhanced under illumination of the subject with special light, for example, the observation mode is switched to the special observation mode. The special light is light high in absorbance for a specific body tissue, for example. A case where the body tissue enhanced in the special observation mode is a superficial blood vessel will be described hereinafter.

Superficial blood vessels include blood having hemoglobin inside. Hemoglobin is known to have absorbance peaks near a wavelength of 415 nm and near a wavelength of 550 nm. Therefore, by irradiating the subject with special light suitable for enhancing superficial blood vessels (specifically, light with high intensity near 415 nm that is a peak of the absorbance of hemoglobin), it is possible to obtain a shot image where the superficial blood vessels have been enhanced. Also, by irradiating the subject with special light with high intensity near 550 nm that is the other peak of the absorbance of hemoglobin, together with the light near 415 nm, it is possible to obtain a bright shot image while maintaining the state of the superficial blood vessels enhanced. Note that, in observing the superficial blood vessels, the peak of the spectral intensity of the special light does not necessarily need to completely correspond with 415 nm, but the special light needs only to contain light having a wavelength of 415 nm. For example, in consideration of the standpoints such as the easiness of manufacture, the stability of product performance, and stable supply of products, it is preferred to select light having a peak wavelength in a range of wavelengths of 405 nm to 425 nm.

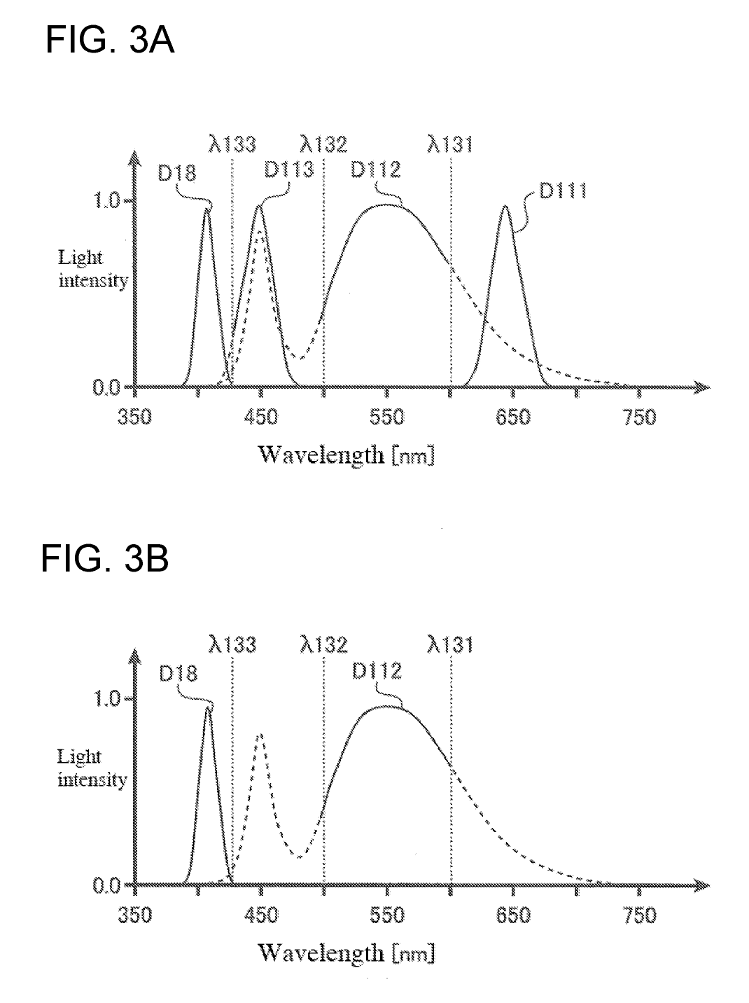

FIGS. 3(a) and 3(b) show the spectral intensity distributions of the illumination light L emitted from the electronic scope 100 in the observation modes: FIG. 3(a) shows the spectral intensity distribution of the illumination light L in the normal observation mode (normal light), and FIG. 3(b) shows the spectral intensity distribution of the illumination light L in the special observation mode (special light). In the spectral intensity distributions shown in FIG. 3, the x-axis represents the wavelength (nm) and the y-axis represents the intensity of the illumination light L. Note that the y-axis shows normalized intensity where the maximum value is 1.

When the electronic endoscope system 1 is in the normal observation mode, the first to third light source units 111 to 113 and the LED 18 are driven to emit light, but the fourth light source unit 114 is not driven. In FIG. 3(a), shown are intensity distributions D111 to D113 and D18 of light emitted from the first to third light source units 111 to 113 and the LED 18, respectively. Also, in FIGS. 3(a) and 3(b), shown by the dotted lines are cut-off wavelengths A131 to A133 of the dichroic mirrors 131 to 133. Among the spectral intensity distributions shown in FIG. 3(a), the regions shown by the solid lines are regions of light emitted from the electronic scope 100 and used as the illumination light L. The regions shown by the broken lines are regions of light that is not emitted from the light source device 201 and unused as the illumination light L.

The spectral intensity distribution D111 of the light emitted from the first light source unit 111 is a steep intensity distribution having a peak at a wavelength of about 650 nm. The spectral intensity distribution D112 of the light emitted from the second light source unit 112 has two peaks at wavelengths of about 450 nm and about 550 nm, which are a peak of the spectral intensity distribution of the light emitted from the blue LED 112 and a peak of the spectral intensity distribution of the fluorescence generated by the green fluorescent material, respectively. The spectral intensity distribution D113 of the light emitted from the third light source unit 113 is a steep intensity distribution having a peak at a wavelength of about 450 nm. The spectral intensity distribution D18 of the light emitted from the LED 18 is a steep intensity distribution having a peak at a wavelength of about 415 nm.

In the normal observation mode, the illumination light L having a wide wavelength band ranging from the ultraviolet region (part of the near-ultraviolet region) to the red region is emitted from the electronic scope 100. The spectral intensity distribution of this illumination light L is a combination of the regions shown by the solid lines among the spectral intensity distributions D111 to D113 and D18 shown in FIG. 3(a). By shooting the subject using this illumination light L, a normal color shot image can be obtained.

Note that, in the normal observation mode, the LED 18 does not necessarily need to be driven to emit light. Even when the LED 18 is not driven, the illumination light L having a wide wavelength band ranging from blue to red regions is emitted. By shooting the subject using this illumination light L, a normal color shot image can be obtained.

When the electronic endoscope system 1 is in the special observation mode, the second light source unit 112 and the LED 18 are driven to emit light, but the first, third, and fourth light source units 111, 113, and 114 are not driven. By doing so, the intensity near a wavelength of 415 nm that is a peak of the absorbance of hemoglobin is relatively high compared to the intensity in the other wavelength bands, whereby a shot image where the superficial blood vessels have been enhanced can be obtained. Also, the light emitted from the second light source unit 112 contains a light component near a wavelength of 550 nm that is the other peak of the absorbance of hemoglobin. Therefore, by driving the second light source unit 112 together with the LED 18, the brightness of the shot image can be increased while the state of the superficial blood vessels enhanced is maintained.

As described above, according to this embodiment, the electronic endoscope system 1 has a plurality of light source units 111 to 114 and the LED 18. The light emission of the plurality of light source units 111 to 114 and the LED 18 is individually controlled according to the observation mode. Therefore, by selecting one or ones of the light source units 111 to 114 and the LED 18 that should be driven to emit light and changing the drive current of the selected one or ones, it is possible to switch the spectral intensity characteristic of the illumination light L to one corresponding to the observation mode used.

The electronic endoscope system 1 of this embodiment also has a twin mode, as an observation mode, where shooting is performed while the normal observation mode and the special observation mode can be switched to each other alternately. In the twin mode, the observation mode is alternately switched between the normal observation mode and the special observation mode for each frame of the shot image. Therefore, the emission control of the light source units 111 to 114 and the LED 18 is switched for each frame of the shot device. Specifically, in the normal observation mode, while the first to third light source units 111 to 113 and the LED 18 are driven to emit light, the fourth source unit 114 is not driven. According to an embodiment, the LED 18 is not driven. In the special observation mode, while the second light source unit 112 and the LED 18 are driven to emit light, the first, third, and fourth source units 111, 113, and 114 are not driven. The shot image taken in the normal observation mode (normal shot image) and the shot image taken in the special observation mode (special shot image) are combined in the rear-stage signal processing circuit 28. In this way, the normal shot image and the special shot image are displayed side by side on the monitor 300.

Therefore, according to an embodiment, the light source drive circuits 17 and 141 to 144 are configured to generate control signals for individually controlling the light emission of the LED 18 and the light source device 201. In relation to this, preferably, the electronic scope 100 includes the solid-state imaging device 14 configured to generate an image signal by imaging the subject with a predetermined frame period, and the light source drive circuits 17 and 141 to 144 are configured to generate the first control signal that drives at least the light source device 201 to emit light and the second control signal that drives at least the LED 18 to emit light alternately switching the control signals for each frame of the image signal.

Note that both the light emitted from the LED 18 and the light emitted from the fourth light source unit 114 are light in the violet wavelength band. Therefore, when the subject is to be irradiated with light in the violet wavelength band, only either one of the LED 18 and the fourth light source unit 114 needs to emit light. When the fourth light source unit 114 is driven to emit light, the violet LED light emitted from the fourth light source unit 114 passes through the LCB 11 to irradiate the subject. While the LCB 11 has a characteristic of transmitting visible light, the transmittance thereof varies with the wavelength band: e.g., as the wavelength of light is shorter, the transmittance is smaller. Also, the LCB 11 has a shape of which the length is 100 m or longer from the connecting portion 102 of the electronic scope 100 to the tip portion of the insertion tube 101. Therefore, assuming that the light amount of the violet LED light incident to the LCB 11 is 100%, the light amount of the violet LED light output from the tip portion of the insertion tube 101 through the LCB 11 will be reduced to approximately 40%. This reduces the amount of the violet LED light for irradiation of the subject, darkening the shot image in some cases. On the contrary, when the LED 18 placed in the tip portion of the insertion tube 101 is driven to emit light in place of the fourth light source unit 114, the violet LED light emitted from the LED 18 causes no loss in light amount due to the transmission through the LCB 11, and thus any shortage in the light amount of the violet LED light for irradiation of the subject can be prevented.

Therefore, according to an embodiment, the light source drive devices 17 and 141 to 144 are configured to generate control signals for individually controlling the light emission of the LED 18 (light emitting device) and the light source device 201 according to each of the plurality of modes: preferably, the light source drive circuits 17 and 141 to 144 are configured to generate the first control signal that drives at least the light source device 201 to emit light in the first mode and generate the second control signal that drives at least the LED 18 to emit light in the second mode, thereby controlling the light source device 201 and the LED 18.

In this way, it is possible to solve the problem that the amount of illumination light in the second mode becomes extremely small compared to the amount of illumination light in the first mode.

Also, in this embodiment, the LED 18 that emits light in the violet wavelength band having a comparatively short wavelength, out of the visible light, is placed in the tip portion of the insertion tube 101, and the light source units 111 to 113 that emits the other range of light having a comparatively long wavelength are placed in the light source device 201 of the processor 200. The LCB 11 has a comparatively high transmittance for blue light, green light, and red light longer in wavelength than violet light. Therefore, even though the light source units 111 to 113 are placed in the light source device 201, loss in the amount of the light emitted from the light source units 111 to 113 occurs less easily in the LCB 11.

While the light source device 201 shown in FIG. 2 has the fourth light source unit 114 having a violet LED, the embodiment of the present invention is not limited to this configuration. When the electronic scope 100 has the LED 18, the light source device 201 does not necessarily need to have the light source unit 114. However, since any type of electronic scope 100 is removably connected to the processor 200, the light source device 201 of the processor 200 desirably has the fourth light source unit 114 in case that the electronic scope 100 having no LED 18 may be connected to the processor 200 and used.

### Second Embodiment

Next, the electronic endoscope system 1 according to the second embodiment of the present invention will be described. The electronic endoscope system 1 according to the second embodiment is the same as the electronic endoscope system 1 of the first embodiment except that the configuration of the light source device 201 of the processor 200 is different from that of the first embodiment.

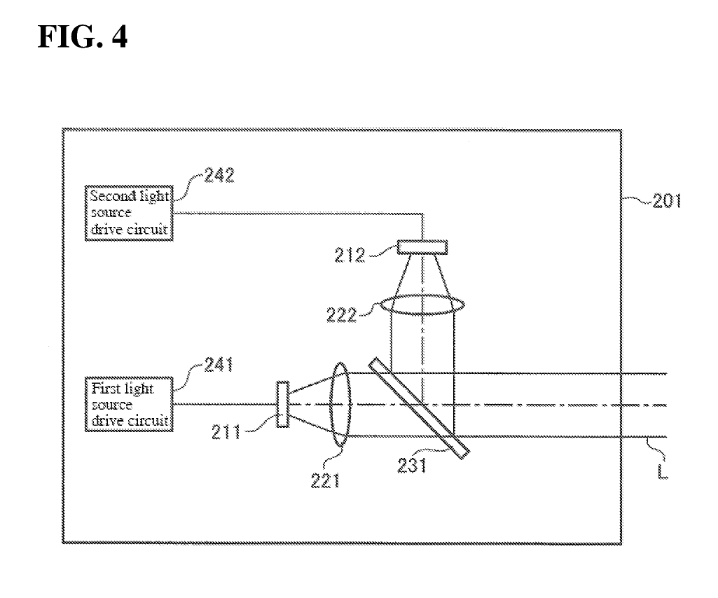

FIG. 4 is a block diagram of the light source device 201 included in the processor 200 of the electronic endoscope system 1 of the second embodiment. The light source device 201 includes first and second light source units 211 and 212. Light emission of the first and second light source units 211 and 212 is individually controlled with control signals generated by first and second light source drive circuits 241 and 242, respectively.

The first light source unit 211 is a red light emitting diode (LED) that emits light in the red wavelength band (e.g., wavelengths of 620 to 680 nm). The second light source unit 212 has a blue LED that emits light in the blue wavelength band (e.g., wavelengths of 430 to 470 nm) and a fluorescent material. The fluorescent material is excited by the blue LED light emitted from the blue LED, to emit fluorescence in the green wavelength band (e.g., wavelengths of 460 to 600 nm).

Collimator lenses 221 and 222 are placed forward of the light source units 211 and 212, respectively, in the light emitting direction. The red LED light emitted from the first light source unit 211 is converted to parallel light by the collimator lens 221 and incident to a dichroic mirror 231. The light emitted from the second light source unit 212, i.e., the blue LED light and the green fluorescence are converted to parallel light by the collimator lens 222 and incident to the dichroic mirror 231. The dichroic mirror 231 combines the light path of the light emitted from the first light source unit 211 and the light path of the light emitted from the second light source unit 212. Specifically, the dichroic mirror 231, having a cut-off wavelength near 600 nm, has a characteristic of transmitting light having a wavelength longer than or equal to the cut-off wavelength and reflecting light having a wavelength shorter than the cut-off wavelength. Therefore, while the red LED light emitted from the first light source unit 211 passes through the dichroic mirror 231, the light emitted from the second light source unit 212 is reflected from the dichroic mirror 231. In this way, the light path of the red LED light and the light path of the blue LED light and the green fluorescence are combined. The light of which the light paths have been combined by the dichroic mirror 231 is emitted from the light source device 201 as illumination light L.

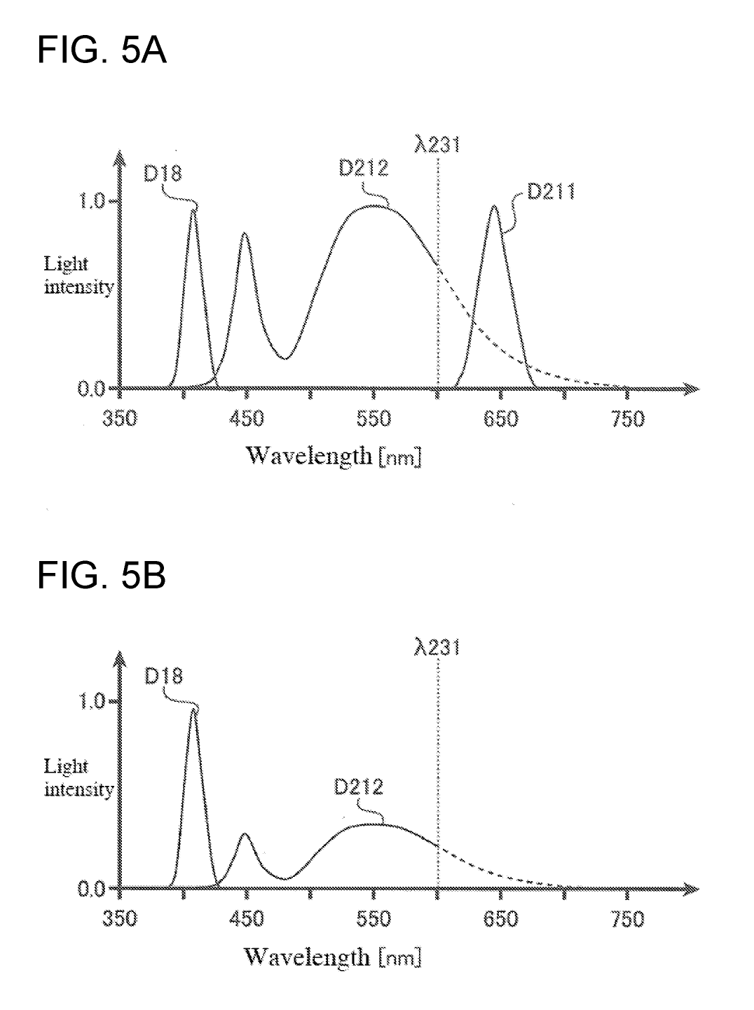

FIG. 5 shows the spectral intensity distributions of the illumination light L emitted from the electronic scope 100 in the observation modes: FIG. 5(a) shows the spectral intensity distribution of the illumination light L in the normal observation mode (normal light), and FIG. 5(b) shows the spectral intensity distribution of the illumination light L in the special observation mode (special light). In the spectral intensity distributions shown in FIG. 5, the x-axis represents the wavelength (nm) and the y-axis represents the intensity of the illumination light L. Note that the y-axis shows normalized intensity where the maximum value is 1.

When the electronic endoscope system 1 is in the normal observation mode, the first and second light source units 211 and 212 and the LED 18 are driven to emit light. In FIG. 5(a), shown are spectral intensity distributions D211, D212, and D18 of light emitted from the first and second light source units 211 and 212 and the LED 18, respectively. Also, in FIG. 5(a), a cut-off wavelength Δ231 of the dichroic mirror 231 is shown by the dotted line. Among the spectral intensity distributions shown in FIG. 5(a), the regions shown by the solid lines are regions of the light emitted from the electronic scope 100 and used as the illumination light L.

The light paths of the light emitted from the light source units 211 and 212 are combined by the dichroic mirror 231, and the LED 18 is driven to emit light, whereby the illumination light L having a wide wavelength band ranging from the ultraviolet region (part of the near-ultraviolet region) to the red region (normal light) is emitted from the electronic scope 100. The spectral intensity distribution of the illumination light L (normal light) is a combination of the regions shown by the solid lines among the spectral intensity distributions D211, D212, and D18 shown in FIG. 5(a). By irradiating the subject with this illumination light L, a normal color shot image can be obtained.

When the electronic endoscope system 1 is in the special observation mode, the second light source unit 212 and the LED 18 are driven to emit light, but the first light source unit 211 is not driven. Also, the drive current of the second light source unit 212 is set to be smaller than that in the normal observation mode. By doing so, the intensity near a wavelength of 415 nm that is a peak of the absorbance of hemoglobin, out of the illumination light L (special light), is relatively high compared to the intensity in the other wavelength bands, whereby a shot image where superficial blood vessels have been enhanced can be obtained. Also, the light emitted from the second light source unit 212 contains light near a wavelength of 550 nm that is the other peak of the absorbance of hemoglobin. Therefore, by driving the second light source unit 212 to emit light together with the light source unit 211, the brightness of the shot image can be increased while the state of the superficial blood vessels enhanced is maintained.

### Third Embodiment

Next, the electronic endoscope system 1 according to the third embodiment of the present invention will be described. The electronic endoscope system 1 according to the third embodiment is different from those of the first and second embodiments in that the light source device 201 has an optical filter 351 that transmits only light in a specific wavelength band.

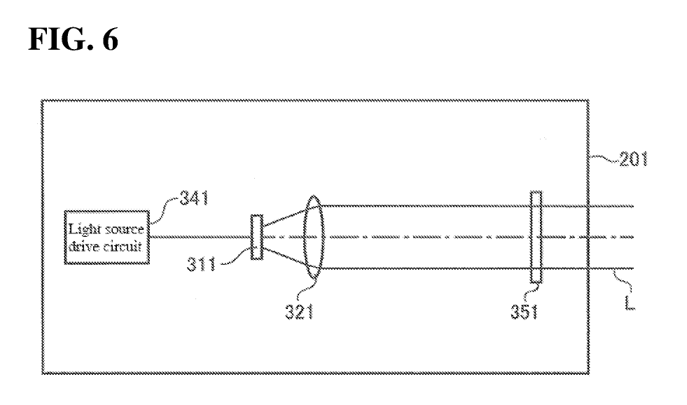

FIG. 6 is a block diagram of the light source device 201 included in the processor 200 of the electronic endoscope system 1 of the third embodiment. The light source device 201 includes a light source unit 311. Light emission of the light source unit 311 is controlled with a control signal generated by a light source drive circuit 341. The light source unit 311 has a blue LED that emits light in the blue wavelength band (e.g., wavelengths of 430 to 470 nm) and a fluorescent material. The fluorescent material is excited by the blue LED light emitted from the blue LED, to emit fluorescence in the green wavelength band (e.g., wavelengths of 460 to 600 nm). The blue LED light and the green fluorescence are mixed to emit pseudo-white light from the light source unit 311. The light emitted from the light source unit 311 is converted to parallel light by a collimator lens 321.

The light source device 201 also has an optical filter 351 insertable to and extractable from the light path of the light emitted from the light source unit 311. The optical filter 351 has a filter characteristic of transmitting only light in a wavelength band near 550 nm.

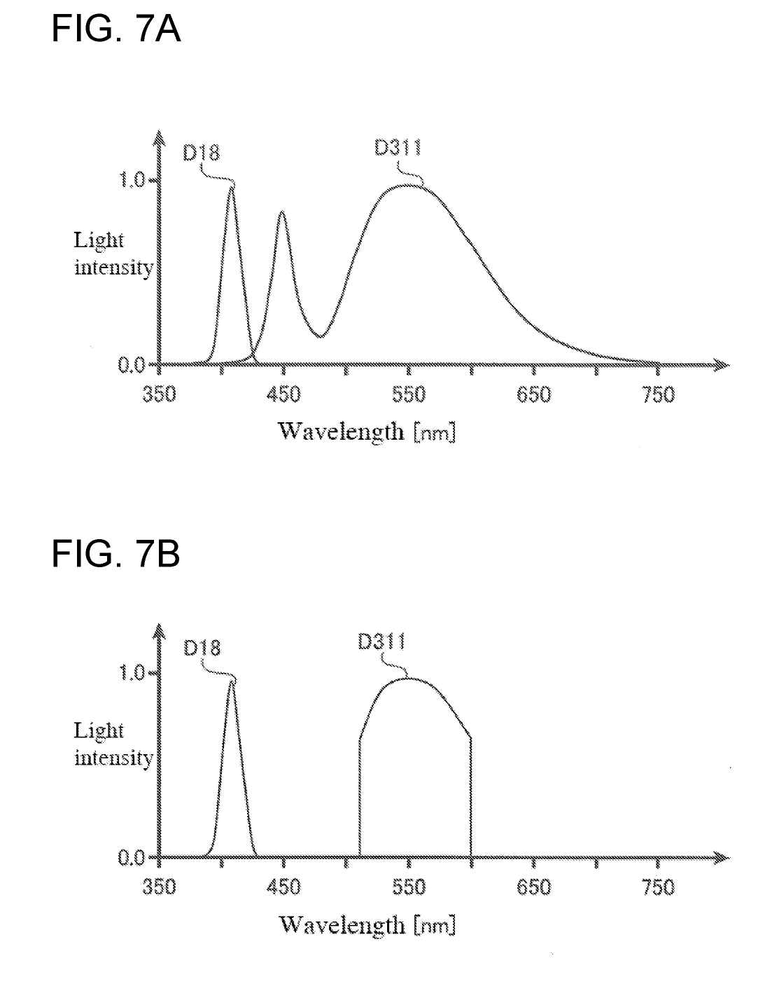

FIG. 7 shows the spectral intensity distributions of illumination light L emitted from the electronic scope 100 in the observation modes: FIG. 7(a) shows the spectral intensity distribution of the illumination light L in the normal observation mode (normal light), and FIG. 7(b) shows the spectral intensity distribution of the illumination light L in the special observation mode (special light). The x-axis of the spectral intensity distributions in FIG. 7 represents the wavelength (nm) and the y-axis represents the intensity of the illumination light L. Note that the y-axis shows normalized intensity where the maximum value is 1.

When the electronic endoscope system 1 is in the normal observation mode, the light source unit 311 and the LED 18 are driven to emit light, and the optical filter 351 is extracted from the light path. A spectral intensity distribution D311 of the light emitted from the light source unit 311 has peaks at wavelengths of about 450 nm and about 550 nm. The two peaks are peaks of the spectral intensity distributions of the blue LED light and the green fluorescence. A spectral intensity distribution D18 of the light emitted from the LED 18 has a peak at a wavelength of 415 nm.

When the electronic endoscope system 1 is in the special observation mode, the light source unit 311 and the LED 18 are driven to emit light. Also, the optical filter 351 is inserted onto the light path. Therefore, the light emitted from the light source unit 311 is restricted to light having the intensity only in a wavelength band near 550 nm. In this way, the intensity near a wavelength of 415 nm and near a wavelength of 550 nm that are peaks of the absorbance of hemoglobin is relatively high compared to the intensity in the other wavelength bands, whereby a shot image where superficial blood vessels have been enhanced can be obtained.

The light source device 201 is included in the processor 200 in the first to third embodiments. According to an embodiment, it is also preferable to place the light source device 201 in the electronic scope 100. In this case, the light source device 201 may be placed in an operation portion with which an operator operates the electronic scope 100, located between the connecting portion 102 and the tip portion 101A. In this case, also, the light emitted from the light source device 201 (first light) is incident to the LCB 11 and guided through the LCB 11 to the tip portion 101A.

Also, according to an embodiment, it is preferable to place the light source device 201 as a component device of the electronic endoscope system 1 separate from the processor 200.

Note that, in this embodiment, the light source unit 311 is not limited to an LED having a fluorescent material. For example, the light source unit 311 may be a lamp emitting white light, such as a xenon lamp.

While illustrative embodiments of the present invention have been described, the embodiments of the present invention are not limited to those described above, but various modifications are possible within the technical ideas of the present invention. For example, appropriate combinations of the illustratively expressed embodiments in this description, obvious embodiments, etc. are also included in the embodiments of the present invention.

For example, while the light source units each have an LED in the above embodiments, the present invention is not limited to this. A laser diode (LD) may be used for each light source unit. Also, it is possible to use an LD in place of the LED 18 placed in the tip portion of the insertion tube 101.

While one LED 18 is provided in the tip portion of the insertion tube 101 in the above embodiments, the present invention is not limited to this. For example, a plurality of LEDs 18 may be placed in the tip portion 101A of the insertion tube 101. In this case, according to an embodiment, the kinds of light emitted from the LEDs 18 placed in the tip portion 101A should preferably be light in a wavelength band for which the light transmittance of the LCB 11 is less than or equal to that for the light emitted from the light source device 201, because, with such light, the reduction in light amount can be efficiently minimized.

### DESCRIPTION OF REFERENCE NUMERALS

1 Electronic endoscope system
11 LCB
12 Light distribution lens
13 Objective lens
14 Solid-state imaging device
15 Driver signal processing circuit
16 Memory
17 Light source drive circuit
18 LED
19 Light distribution lens
21 System controller
22 Timing controller
23 Memory
24 Operation panel
25 Condenser lens
26 Front-stage signal processing circuit
27 Image memory
28 Rear-stage signal processing circuit
100 Electronic scope
101 Insertion tube
101A Tip portion
101B, 101C Emission port
102 Connecting portion
111 to 114 Light source unit
121 to 124 Collimator lens
131 to 133 Dichroic mirror
141 to 144 Light source drive circuit
200 Processor
201 Light source device
211, 212 Light source unit
221, 222 Collimator lens
231 Dichroic mirror
241, 242 Light source drive circuit
311 Light source unit
321 Collimator lens
341 Light source drive circuit
351 Optical filter

1. An electronic scope comprising:
an insertion tube configured to be inserted into a body cavity and provided with a light emission port in its tip portion;
a light guide configured to guide light down to the tip portion of the insertion tube so as to emit first light from the tip portion; and
a light emitting device configured to emit second light from the tip portion, the light transmittance of the light guide for a wavelength band of the second light being less than or equal to the transmittance for a wavelength band of the first light,
wherein the light path length of the second light from the light emitting device to an emission port for the second light placed in the tip portion is shorter than the light path length of the first light in the light guide.

2. The electronic scope of claim 1, wherein the light emitting device is placed in the tip portion.

3. The electronic scope of claim 1, wherein the second light has a peak wavelength between a wavelength of 405 nm and a wavelength of 425 nm.

4. The electronic scope of claim 1, wherein a plurality of solid-state light emitting devices are placed in the tip portion, and the light emitting device is one of the plurality of solid-state light emitting devices.

5. The electronic scope of claim 1, comprising a light source device configured to emit the first light to the light guide.

6. An electronic endoscope system comprising:
an electronic scope comprising:
an insertion tube configured to be inserted into a body cavity and provided with a light emission port in its tip portion;
a light guide configured to guide light down to the tip portion of the insertion tube so as to emit first light from the tip portion; and
a light emitting device configured to emit second light from the tip portion, the light transmittance of the light guide for a wavelength band of the second light being less than or equal to the transmittance for a wavelength band of the first light,
wherein the light path length of the second light from the light emitting device to an emission port for the second light placed in the tip portion is shorter than the light path length of the first light in the light guide; and
an electronic endoscope processor to which the electronic scope is removably connectable,
wherein the electronic endoscope processor includes
a light source device configured to emit the first light, and
a light source drive circuit configured to generate a control signal that controls light emission of the light emitting device and the light source device.

7. The electronic endoscope system of claim 6, wherein the electronic endoscope processor has an optical filter insertable to and retractable from the light path of the first light.

8. The electronic endoscope system of claim 7, wherein the optical filter has a filter characteristic of transmitting only light in a green wavelength band out of a visible light band.

9. An electronic endoscope system comprising:
an electronic scope comprising:
an insertion tube configured to be inserted into a body cavity and provided with a light emission port in its tip portion;
a light guide configured to guide light down to the tip portion of the insertion tube so as to emit first light from the tip portion; and
a light emitting device configured to emit second light from the tip portion, the light transmittance of the light guide for a wavelength band of the second light being less than or equal to the transmittance for a wavelength band of the first light,
wherein the light path length of the second light from the light emitting device to an emission port for the second light placed in the tip portion is shorter than the light path length of the first light in the light guide; and
a light source device configured to emit the first light to the light guide.

10. The electronic endoscope system of claim 6, wherein the first light includes light having a wavelength longer than the second light.

11. The electronic endoscope system of claim 6, wherein the light source device has a plurality of light source units configured to emit light different in wavelength band from each other.

12. The electronic endoscope system of claim 11, wherein one of the plurality of light source units is a light source unit configured to emit the second light.

13. The electronic endoscope system of claim 6, comprising a light source drive circuit configured to generate a control signal for individually controlling light emission of the light emitting device and the light source device according to each of a plurality of modes,
wherein the light source drive circuit is configured to generate a first control signal that drives at least the light source device to emit light in a first mode and generate a second control signal that drives at least the light emitting device to emit light in a second mode, thereby controlling the light source device and the light emitting device.

14. The electronic endoscope system of claim 6, comprising a light source drive circuit configured to generate a control signal for individually controlling light emission of the solid-state light emitting devices and the light source device,
wherein the electronic scope includes an imaging device configured to image a subject with a predetermined frame period to generate an image signal, and
the light source drive circuit is configured to generate a first control signal that drives at least the light source device to emit light and a second control signal that drives at least the light emitting device to emit light alternately switching the control signals for each frame of the image signal.
